(19) United States
(12) Patent Application Publication
(10) Pub. No.: US 2009/0053784 A1
(43) Pub. Date: Feb. 26, 2009

(54) EXPRESSION OF AN ACTIVE CARRIER FROM XYLOSE IN GENETICALLY MODIFIED SACCHAROMYCES CEREVISAE

(76) Inventors: Maria Jose TRAVASSOS LEANDRO, Monte da Caparica (PT); Paula Maria Theriaga Mendes Bernardo Goncalves De Zoeten, Lisboa (PT); Isabel Maria Spencer Viera Martins, Alges (PT); Augusto Martins, Alges (PT)

Correspondence Address: SUGHRUE MION, PLLC, 2100 PENNSYLVANIA AVENUE, N.W., SUITE 800, WASHINGTON, DC, 20037, US

(21) Appl. No.: 12/025,625

(22) Filed: Feb. 4, 2008

Related U.S. Application Data
(63) Continuation of application No. PCT/PT2006/000021, filed on Aug. 4, 2006.

(30) Foreign Application Priority Data
Aug. 5, 2005 (PT) 103331

(51) Int. Cl. C12P 7/06; C12N 15/11; C12N 15/00; C12N 1/19

(52) U.S. Cl. 435/161; 536/23.5; 435/320.1; 435/254.2; 435/254.21

(57) ABSTRACT

The present invention confers to the ferementative yeast Saccharomyces cerevisiae, genetically modified by insertion of a nucleic acid sequence encoding a xylose and a glucose active transporter, the ability to assimilate xylose using a system of co-transport with protons exhibiting a high affinity for xylose. The invention is useful for the production of bioethanol from plant biomass and other lignocellulosic materials, using genetically modified microorganisms for assimilating and fermenting xylose in mixtures of hexoses and pentoses resulting from raw material of industrial interest.

This is a continuation of International Application No. PCT/PT2006/000021, filed Aug. 4, 2006, which claims priority to Portuguese Patent Application No. 103331, filed Aug. 5, 2005, all of which are hereby incorporated by reference.

### OBJECT OF THE INVENTION

The present invention refers to the modified yeast, preferably Saccharomyces cerevisiae, with the introduction of a novel gene corresponding to an active transporter for xylose. It is also object of the present invention the co-transport of xylose/proton by yeasts in the presence of glucose. Another object of the present invention is the use of recombinant yeasts, with the same xylose transporting system, in the fermentation of lignocellulosic hydrolysates.

The object of the present invention is to provide to the bioethanol fuel industry yeasts capable of assimilating faster xylose in glucose mixtures and to ferment xylose more efficiently and with higher specific productivity.

### STATE OF THE ART

Action programmes worldwide target to the production of biofuels, with relevance to bioethanol, as an alternative and renewable energy. Those measures aim to reduce the dependency on petroleum and to reduce the emission of gases and the resulting climatic changes. At present, crops and other substrates from agricultural origin rich in glucose are used in the industrial production of ethanol by the yeast Saccharomyces cerevisiae. The lignocellulosic materials are the most abundant components of plant biomass. They make up the major forest product and a considerable fraction of waste resulting from agricultural practice. The development of processes for its bioconversion into ethanol is potentially important and strongly stimulated.

Cellulose in lignocellulosic materials is a polymer exclusively formed by glucose, whilst the hemicelluloses fraction is composed of polymers containing a mixture of hexoses (glucose, galactose and mannose) and of pentoses (xylose, arabinose and ribose). Xylose is the principal pentose present in the hemicelluloses, composing 17% to 31% of its dry weight. About 80% of the total xylose can be recovered as fermentable sugar in the hemicellulosic hydrolysates. The use of lignocellulosic materials for a cost-effective production of ethanol by Saccharomyces requires the total fermentation of xylose. This yeast, however, does not present a natural ability to convert xylose into ethanol. There are other yeasts capable of fermenting xylose, but the hemicellulosic hydrolysates contain several compounds such as organic acids, furans and phenols inhibiting the fermentation process. Therefore S. cerevisiae is the only known microorganism capable of fermenting effectively in this stressful environment (Olsson and Hahn-Hägerdal, “Fermentation of lignocellulosic hydrolysates for ethanol production”, Enzyme Microbial Technol. 18: 312-331, 1996).

Recombinant strains of S. cerevisiae have been produced in which two genes for xylose catabolism were inserted: xylose reductase (XR), which reduces xylose to xylitol, and xylitol dehydrogenase (XDH), oxidizing xylitol to xylulose. This compound is already naturally metabolized by S. cerevisiae following the pentose phosphate pathway and the glycolytic pathway for ethanol production. The genes for the XR and XDH enzymes were obtained from the yeast Pichis stipitis, which naturally ferments xylose. With these genes, S. cerevisiae metabolizes xylose, but does not produce ethanol in significant concentrations. In this yeast, xylulose is phosphorylated to xylulose-5-phosphate by means of a xylulose kinase (XK). The XK native gene was over-expressed in S. cerevisiae strains containing heterologous XR and XDH. The novel gene combination was object of chromosomal integration for producing strains with a stable phenotype and amenable to cultivation in industrial substrates (W09742307A1). The resulting strains produce significant ethanol concentrations, but with low productivity values.

Several strategies have been followed for improving the productivity in ethanol production from xylose by recombinant S. cerevisiae strains. Three of these strategies succeeded. One consisted in subjecting the S. cerevisiae recombinants to random mutagenesis, using EMS (ethyl methane sulphonate) as mutagenic agent, and selecting the obtained mutants for a more effective fermentation (US 2003/0157675 A1). Another approach subjected the recombinant strains to a strong selective stress, using continuous culture on chemostat and anaerobiosis, for selection of the most suitable ones for fermenting xylose (W003078643). The third strategy used the xylose catabolic pathway occurring usually in bacteria. In this group of microorganisms, the xylose is transformed directly into xylulose by means of a xylose isomerase (XI). The successive attempts to express XI of bacterial origin in S. cerevisiae had failed. Recently, a XI of fungal origin was isolated and expressed in S. cerevisiae (W003062430).

This document describes host cells transformed with a nucleic acid sequence encoding a xylose isomerase obtained from a filamentous fungus. This sequence confers to the host cell the ability to improve xylose metabolism. This improvement can also possibly be achieved by the expression of a heterologous pentose transporter. In fact, this document only discloses the isolation and cloning of a gene encoding a xylose isomerase and only considers the improvement in host cells of xylose metabolism by the hypothetical expression of a heterologous pentose transporter.

However, the productivities obtained in the production of ethanol from xylose, using the best strains available, is still inferior when compared to the ones obtained when the yeast ferments glucose. One possible obstacle for obtaining higher values is found when xylose enters the cell (Hahn-Hägerdal et al, “Metabolic engineering of Saccharomyces cerevisiae for xylose utilization”, Adv Biochem. Eng/Biotechnol. 73: 53-84, 2001; Jeffries and Jin, “Metabolic engineering for improved fermentation of pentose by yeasts”, Appl. Microbiol. Biotechnol. 63: 495-509, 2004).

Xylose is a weak substrate of the transporters mediating the fast entrance of glucose and other hexoses in S. cerevisiae. HXT transporters present an affinity towards xylose one or two times lower than towards glucose. Consequently, in the presence of glucose, xylose is not assimilated. In the absence of glucose, xylose assimilation and, consequently, the fermentative ability are also reduced. It is conceivable that the expression of transporters with higher affinity towards xylose, namely the ones transporting xylose through active transport mechanisms of the proton symport type, enable a more efficient production of ethanol. The energy consumption for transporting xylose into the cell may be translated, in strains with a xylose/proton symport, into a lower biomass yield, increasing concomitantly the specific productivity for ethanol production.

Among the yeasts capable of growing naturally in xylose, Candida intermedia PYCC 4715 stands out due to its high specific growth rate. It has been shown that this yeast produces two transport systems for xylose, one of the facilitated diffusion type and the other of the xylose/proton symport type, presenting the latter a higher affinity for xylose and being only produced when the xylose concentration was relatively low (Gárdony et al, “High capacity xylose transport in Candida intermedia PYCC 4715”, FEMS Yeast Res. 3: 45-52, 2003).

This yeast was considered adequate for isolating the gene of an active xylose transporter (GXS 1) to be expressed in S. cerevisiae. However, it is also mentioned that the three genes responsible for xylose uptake found and isolated so far from natural xylose-utilizing yeasts showed low affinity for xylose. In fact, all the results presented in this document refer not to genes or the corresponding enzymatic activities but to the ability to use xylose and to kinetic characteristics of xylose transport in whole yeast cells.

Despite the progress, the recombinant yeasts developed until now do not show enough efficiency in ethanol production from xylose. There is a need to improve the state of art for fermenting lignocellulosic materials and to produce bioethanol at the industrial level.

Document “Leandro, Maria et al.—Molecular characterisation of xylose transport in Candida intermedia. Yeast, vol. 20, n° Suppl. 1 (2003-07), pp. S246” discloses molecular approaches to the characterization of xylose transport in C. intermedia. In this document it is possible to read an explicit expression stating that “so far, no gene encoding such type of (active) transporter has been isolated from fungi”. In fact, this document describes an unsuccessful attempt to isolate the xylose symporter gene.

Document “Gardony, Mark et al.—Control of xylose consumption by xylose transport in recombinant Saccharomyces cerevisiae. Biotechnology and Bioengineering, vol. 82, n° 7 (2003-06), pp. 818-824″ describes the control exercised by the step of xylose transport over the xylose consumption rate by transformed S. cerevisiae strains expressing heterologous xylose reductase (XR) and xylitol dehydrogenase (XDH), and overexpressing the native xylulokinase (XK) gene. The main result was that the control was higher at low xylose concentrations, which is not in anyway related to isolation and cloning of genes encoding an active xylose/glucose transporter.

Since the 80's it has been known that yeasts produce active xylose transporters, the respective activities/capacities being regulated by the substrate concentration in the surrounding medium. However, the approaches followed by several groups worldwide to isolate and clone the corresponding gene were unsuccessful, mainly due to the routine experimental protocols followed in similar cases.

The present invention solves this problem developing a method for isolating this gene (responsible for the expression of an active xylose/glucose transporter) and further to clone it into a host cell thereby demonstrating xylose/glucose-H⁺symport activity. The step(s) that allowed the invention to achieve this outcome resulted from a combination of skills is yeast physiology, biochemistry and molecular biology, which is uncommon among those skilled in the art of gene cloning and isolation. In general, the higher activity of the symport at low xylose concentrations, described in document “Gárdony et al, “High capacity xylose transport in Candida intermedia PYCC 4715”, FEMS Yeast Res. 3: 45-52, 2003”, leads researchers to isolate the cDNA based on the increase in the respective mRNA and using RT (Reverse Transcriptase)-PCR and degenerate primers. This standard approach proved to be unsuccessful. However, in the present invention this problem is circumvented by comparing plasma membrane proteins obtained in inducing and repressing growth conditions. This innovative step allowed the isolation of the gene and transformation of the host cell. Demonstration of the presence of the xylose/glucose symporter in the transformed host cells required another innovative step, the co-transformation of both the xylose/glucose facilitator and the active transporter, to put in evidence biphasic kinetics of labelled glucose uptake by cells growing in relatively low glucose medium. This could not be achieved using a host cell transformed solely with the active xylose/glucose transporter due to its very low capacity.

### SUMMARY OF THE INVENTION

Therefore, the problem the present invention aims to solve corresponds to offering a process for a more efficient and cost-effective bioethanol production from lignocellulosic materials.

The solution of this problem is based on the fact that the present inventors were able to identify and isolate a gene encoding an active transporter for xylose/glucose from C. intermedia with a surprisingly high affinity towards xylose in comparison to the transporters that occur naturally in fermentative yeasts. When inserted in a host cell, this gene turns it potentially more effective in consuming and fermenting the xylose present in the mixture of hexoses and pentoses resulting from raw materials of industrial interest for bioethanol production.

Thus, a first aspect of the invention refers to an isolated DNA fragment encoding an active transporter for xylose/glucose, characterized for comprising:

a nucleotide sequence SEQ ID No: 1; or
nucleotide sequence with a homology of at least 80% with the fragment from +1138 to +1315 of the SEQ ID No:1,
or its complementary sequences.

In a second aspect, the invention refers to a cDNA molecule, characterized for comprising:

a nucleotide sequence SEQ ID No: 1; or
a nucleotide sequence with a homology of at least 80% with the fragment from +1138 to +1315 of the SEQ ID No:1.,
or its complementary sequences.

In a third aspect, the invention refers to a plasmid, characterized for comprising a DNA fragment according to claim 1.

In a fourth aspect, the invention refers to a host cell characterized for being transformed with the plasmid according to claim 3, in order to allow the host cell to express the mentioned xylose/glucose active transporter.

In a last aspect, the invention refers to the use of a host cell transformed for ethanol production by means of xylose fermentation from a medium comprising a xylose source.

### BRIEF DESCRIPTION OF THE FIGURES

FIG. 1: Denaturing polyacrylamide gel electrophoresis (10% T) of 20 μg total proteins of plasma and mitochondrial membranes isolated from C. intermedia cells cultivated in 0.5% xylose (X), 2% glucose (G) and 4% xylose (4×). The gel was stained with Coomassie Blue. M—Sigma Marker (Wide Range), p—plasma membranes; n—mitochondrial membranes.

FIG. 2: Amino acid sequence from the N-terminal region of the Gxs1p protein and degenerated primers designed from this region.

FIG. 3: Northern Blot analysis of the GXS1 gene expression. Total RNA was isolated from C. intermedia PYCC 4715 cultures in Verduyn medium containing 0.5% xylose (X), 2% glucose (G) or 4% xylose (4×) as single carbon and energy source. Each sample contains 10 μg of total RNA, separated in a denaturating 1.2% agarose gel and subsequently transferred to a nylon membrane (Hybond-N). A 300 bp fragment, amplified by means of CiGXSL1 and CiGXSR3 primers, was used as specific probe for the GXS1 gene. A 172 bp fragment from the actin gene was amplified using the ActCiL1 (5′-AACAGAGAGAAGATGACCCAGA; SEQ ID NO:2) primer and the ActCiR1 (5′-GCAAAGAGAAACCAGCGTAAA; SEQ ID NO:3) primer and genomic DNA from C. intermedia PYCC 4715 as template. The probes were labelled with [α-³²P]-ATP (Amersham Bioscience) using Prime-a-Gene Labelling System (Promega). Hybridizations and washings were performed as described by Griffioen et al (1996).

FIG. 4: Nucleotide sequence of the GXS1 gene (SEQ ID No. 1), from the first (ATG) to the last (TAA) codon. The sequence +1138 until +1315 is shadowed.

FIG. 5: Extracellular alkalinisation elicited by the addition of xylose (X) or glucose (G) to an aqueous suspension of cells of the MJY2 strain cultivated in mineral medium with 2% (w/v) of glucose.

FIG. 6: Eadie-Hofstee representation of the initial transporter velocities of D-[¹⁴C] xylose (♦) in cells of the MJY2 strain, obtained from a culture in mineral medium with 2% (w/v) of glucose, and of D-[¹⁴C] glucose (□) in cells of the MJY5 strain, cultivated in mineral medium with 2% (w/v) of glucose and 0.05% of maltose.

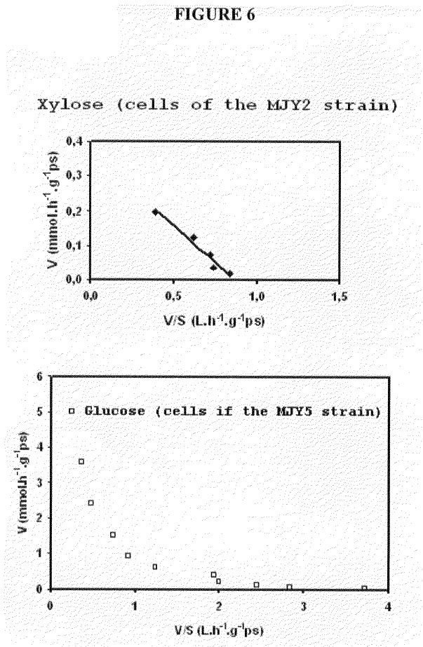

### DETAILED DESCRIPTION OF THE INVENTION

According to a preferred embodiment of the present invention, a process to express in S. cerevisiae a xylose active transporter was developed. This process comprises the insertion of heterologous DNA in yeasts, integrating from that point on a gene for a novel xylose transport system of the xylose/glucose-proton symport type.

Referring to this invention, a process for isolating, cloning and expressing the gene was followed. However, alternative processes may be used by those skilled in the art.

### Identification of the Xylose/Glucose-H+ Active Transporter by SDS-PAGE

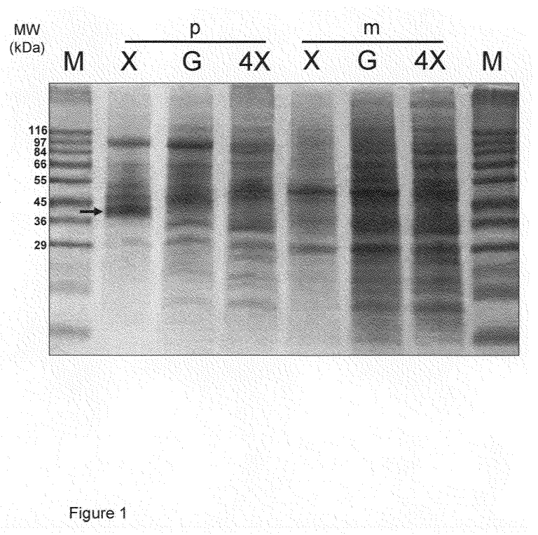

The xylose/glucose active transporter from C. intermedia was identified by comparison of the relative abundance of the proteins present in plasma membranes isolated from C. intermedia cells cultivated under inducing and repressing conditions. With this objective, plasma membranes and mitochondrial membranes were isolated from cells cultivated in Verduyn medium (Verduyn et al, 1992) containing, alternatively, 0.5% of xylose, 2% of glucose or 4% of xylose as single carbon and energy source. The cells were collected in the exponential phase of growth (DO₆₄₀=0.8−2.0) and washed twice with ice-cold distilled water and once with buffer A (0.1 M of glycine, 0.3 M of KCI, pH 7.0). Ten to fifteen grams of cells were then resuspended in 15 ml of buffer A containing 0.1 mM PMSF. The isolation of the membranes was performed from this point on as described by Van Leeuwen et al (1991). With aliquots (20 μg) of the obtained samples, a denaturing polyacrylamide gel electrophoresis in the presence of tricine (Tricine SDS-PAGE; Schlaigger, 1994) was performed. The concentrations of acrylamide and bisacrylamide used in the gel were 10%T and 3%C (% T=total concentration of acrylamide+bisacrylamide and % C=percentage of bisacrylamide relatively to the total). The plasma membrane samples presented a band pattern obviously different from the one presented by the corresponding samples of mitochondrial membranes (FIG. 1) indicating that an efficient separation of the two membrane types occurred. Consequently, it has been found that the observed differences between the band patterns from the plasma membrane samples, corresponding to the different carbon sources, are not a consequence of a contamination by mitochondrial proteins.

The most notable difference between the three plasma membrane samples is indicated by an arrow in FIG. 1. It corresponds to a protein of about 40 kDa molecular weight that seems to be present only in plasma membranes of cells cultivated in 0.5% of xylose. As the molecular weight of this protein is in the expected range for a sugar transporter, it was considered that the band would probably correspond to the xylose/glucose active transporter, kinetically characterized in C. intermedia.

Cloning of the cDNA Encoding the Xylose/Glucose Active Transporter

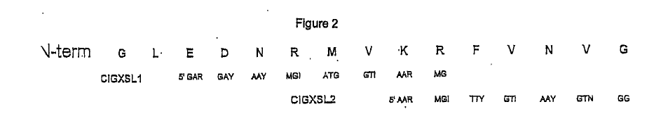

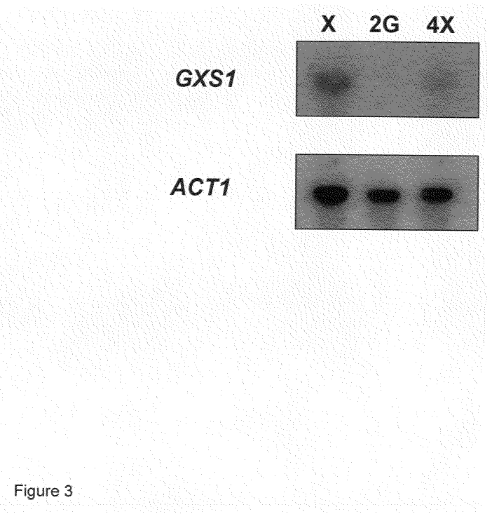

The membrane protein, identified as described, was isolated from a preparative gel loaded with 250 μg of total membrane protein from C. intermedia cells cultivated in 0.5% of xylose. After electrophoresis, the proteins were transferred to a PVDF membrane (Sequi-blot from BIO-RAD). The electrophoresis and the transference were realized according to instructions provided by the manufacturer. The fraction of the membrane containing the protein of interest was cut-off and used for sequencing of the N-terminal end of the protein (Protein Core Facility, Columbia University, USA). The obtained sequence of 15 amino acids is indicated in FIG. 2. From this sequence, degenerated primers were designed (FIG. 2). These primers were used to amplify the cDNA through RACE (Rapid Amplification of cDNA Ends) technique, from total RNA of cells cultivated in 0.5% of xylose. For this purpose, a First Choice RLM-RACE kit (Ambion) was used, according to instructions provided by the manufacturer. The RNA was extracted as described by Griffioen et al (1996) and subsequently purified using RNA cleanup protocol (RNeasy kit, Quiagen). This RNA sample was used as template for the 3′ RACE protocol, in combination with the CiGXSL1 (5′-GARGAYAAYMGIATGGTIAARMG-3′; SEQ ID NO:4) and the CiGXSL2 (5′-AARMGITTYGTIAAYGTNGG-3′; SEQ ID NO:5) primers; I=inosine, Y=C/T, R=A/G, M=A/C and N=A/ T/ or C. Since the design of the primers was based on the sequence of the first amino acids of the protein, it was expected that the 3′ RACE reaction would produce the cDNA almost entirely. In fact, with this reaction a product of about 1.7 kb was obtained, which was cloned in the pMOSBlue vector (Amersham Biosciences) and partially sequenced, using an automatic sequencer ALF Express (Amersham Pharmacia Biotech) and Cy5-labelled primers specific for the vector sequences. The protein encoded by this molecule presented the characteristic properties of a sugar transporter. Next, a Northern blot analysis was performed, which showed that the respective mRNA was abundant in cells cultivated in 0.5% of xylose but was not detectable in cells cultivated in 2% of glucose (FIG. 3).

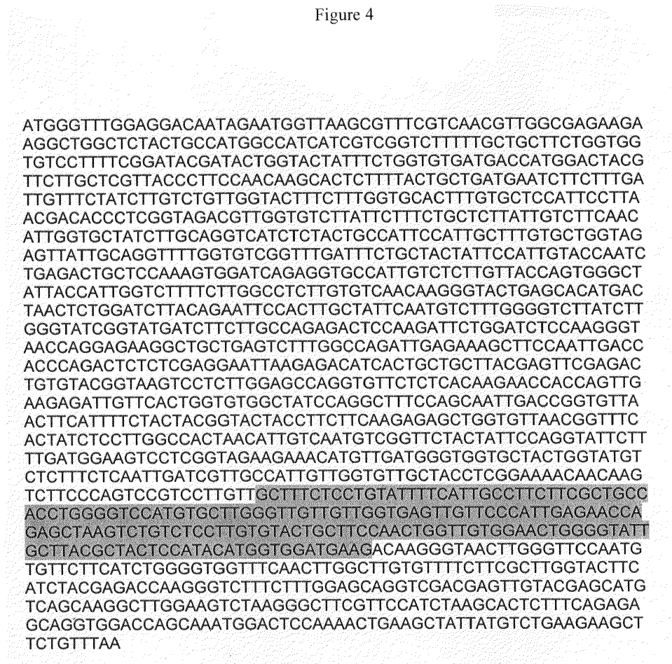

The 5′ end from the cDNA was obtained through the 5′ RACE technique, using the CiGXSR3 (5′-CGTTAAGGAATGGAGCACAAAG-3′; SEQ ID NO:6) primer. The fragments obtained were cloned and sequenced as described in the prior paragraph, showing that an additional amino acid (initializing methionine) and a leader sequence of 28 or 31 amino acids are encoded, indicating the existence of two active sites of transcription initiation. The novel gene was designated GXS1 (Glucose Xylose Symport 1). The correspondent nucleotide sequence (SEQ ID No. 1) is presented in FIG. 4.

Functional Expression in S. cerevisiae

To confirm that the novel transporter encoded by the GXS1 gene was a transporter for glucose and xylose, several plasmids were engineered allowing the expression of the cDNA in S. cerevisiae. A high copy number vector (pMA91; Kingsman et al, 1990), containing the promoter and terminator regions of the PGK1 gene, was used to clone the cDNA from GXS1 in the following way: the total encoding region of the GXS1 gene was amplified by PCR using the GXS1P1 (5′-ATAGCAGATCTCATATGGGTTTGGAGGACAATAGAATG-3′; SEQ ID NO:7) primer and the GXS1P2 (5′-ATAGCAGATCTTCTAGATTAAACAGAAGCRRCTTCAGAC-3′; SEQ ID NO:8) primer. Both primers have a recognition sequence for BglII at the 5′ end and, additionally, they also have recognition sequences for NdeI and XbaI. The pMA91 plasmid was then digested with BglII and ligated with the fragment containing the encoding region from GXS 1, digested with the same enzyme, originating the pPGK-GXS1 plasmid.

A different chimeric gene was engineered using the truncated promoter of the HXT7 gene and was cloned in the YEpLac 195 (multi-copy) and YCpLac 111 (single-copy) vectors (Gietz et al, 1988). A DNA fragment comprising the nucleotides −392 to −1 from the HXT7 promoter was amplified by PCR using the HXT7prom1 (5′-AACCTGCAGCTCGTAGGAACAATTTCGG-3′; SEQ ID NO:9) primer and the HXT7prom2 (5′-GGACGGGACATATGCTGATTAAAATTAAAAAAACTT-3′; SEQ ID NO: 10) primer and the YEpkHXT7 plasmid (Krampe et al, 1998) as template. The fragment was subsequently digested with PstI and NdeI, since the primers contain recognition sites for these enzymes, being afterwards ligated to the YEpLac 195 plasmid, digested with PstI and XbaI, originating the pHGXS1 plasmid. Subsequently, a 0.3 kb fragment containing the terminator region of the PGK gene was amplified using the PGK1 term 1 (5′ -ACCGTGTCTAGATAAATTGAATTGAATTGAATCGATAG-3′; SEQ ID NO:11) primer and the PGK1term2 (5′-TAATTAGAGCTCTCGAAAGCTTTAACGAACGCAGAA-3′; SEQ ID NO:12) primer and the pMA91 plasmid as a template. The primers have at its 5′ ends recognition sites for the XbaI and SacI enzymes, respectively. The fragment containing the terminator region of the PGK gene was subsequently digested with these enzymes and ligated between the XbaI and SacI sites of the pHGXS1 plasmid, originating the pHXT7-GXS1 plasmid.

Finally, the pHXT7-GXS1 plasmid was digested with PstI and SacI generating a fragment containing the total chimeric gene, which was subsequently inserted in the YCplac 111 vector (Gietz et al, 1988), digested with the same enzymes, originating the pHXT7-GXS1 plasmid.

The three plasmids were then used to transform S. cerevisiae TMB 3201 (MATa Δhxt1-17 Δgal2 Δstl1 Δagt1 Δmph2 Δmph3 leu2-3,112 ura3-52 trp1-289 his3-Δ1 ::YIpXR/XDH/XK MAL2-8^{c} SUC2; Hamacher et al, 2002). This strain is not capable of using glucose or xylose as carbon and energy source because it does not express any transport system for these sugars. The transformations originated the MJY2-4 strains: MJY2 (TMB 3201+pHXT7-GXS1), MJY3 (TMB 3201+pPGK-GXS1) and MJY4 (TMB 3201+pHXT7-GXS1-s).

The incapacity of growing in glucose or xylose was overcomed by complementation in both strains containing plasmids with high copy number (MJY2 and MJY3), but the growth in xylose, as single carbon and energy source, was very weak and only in a solid medium culture. The MJY4 strain, containing a plasmid of low copy number, presents a very weak growth in glucose and absence of growth in xylose, suggesting that the occurrence of complementation is dependent on a stronger expression of the gene than the one possible to obtain with this plasmid.

The MJY2 strain was used for investigating the presence of xylose and glucose active transporter. The addition of D-glucose or D-xylose (final concentration of 6.7 mM) to an aqueous suspension of cells (about 30 mg dry weight/ml) of the MJY2 strain, cultivated in YNB medium (Yeast Nitrogen Base) supplemented with 2% (w/v) of glucose, leucine and tryptophan, triggers an increase of the extracellular pH in both cases, indicating the existence of an influx of protons associated to the transport and, therefore, an active transport system co-transporting sugar and H⁺ occurs (FIG. 4). This assay shows that the GXS1 gene encodes a transporter with an active transport mechanism, which accepts as substrate both glucose and xylose.

Kinetics of sugar transport by Gxs1p

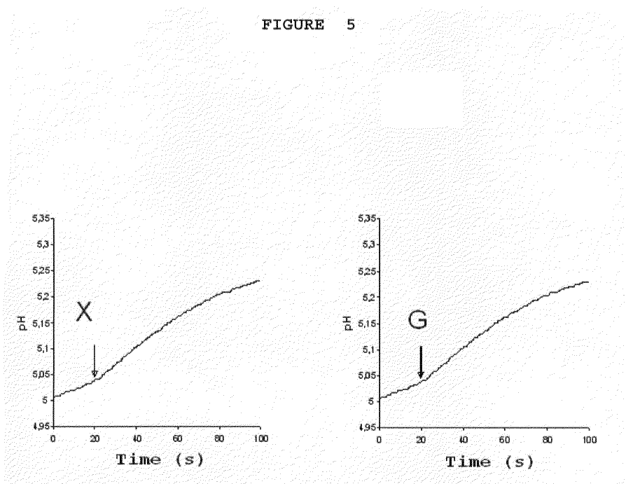

The kinetic constants from transport mediated by Gxs1p were determined in the MJY2 strain, expressing only the active transport system. However, despite its high affinity, the capacity of this transporter does not allow high transport velocities comparable to the facilitated diffusion system. Therefore, in order to give a better sense of the values to be obtained in the kinetic assays with ¹⁴C-D-glucose (Spencer-Martins et al, 1985), substrate for which the affinities of the two transport types differ just in one order of magnitude (facilitated diffusion: Kₘ=2-4 mM; symport: Kₘ=0.2 mM; 25° C., pH 5) instead of two as with xylose (facilitated diffusion: Kₘ=49 mM; symport: Kₘ=0.4 mM; 25° C., pH 5), the MJY5 strain expressing the two transport types present in C. intermedia was used for this purpose. In FIG. 5, an obvious two-phase kinetics for glucose may be observed, indicative for the simultaneous presence of a transport system of the facilitated diffusion type and of the now identified and cloned active transporter of the xylose/glucose-H⁺ symport type, with high relative affinity. The kinetic parameters determined in these conditions in S. cerevisiae were similar to the ones obtained in C. intermedia, origin of the GXS1 gene.

Homology with Other Transporters

The characterization of GXS1 allowed discovering a protein family with some homology towards Gxs1p and which are present in other yeasts (Debaryomyces hansenii, Yarrowia lipolytica and Candida albicans, GenBank accession numbers: CAG86664, EAL01541 and CAG81819, respectively). For none of these proteins is the function known (they are registered in the databases as putative sugar transporters).

1. An isolated DNA fragment encoding an xylose/glucose active transporter, characterized for comprising:
a) a nucleotide sequence SEQ ID No: 1; or
b) a nucleotide sequence with a homology of at least 80% with the fragment from +1138 to +1315 of SEQ ID No: 1, or its complementary sequences.

2. A cDNA molecule, characterized for comprising:
a) a nucleotide sequence SEQ ID No: 1; or
b) a nucleotide sequence with a homology of at least 80% with the fragment from +1138 to +1315 of SEQ ID No:1, or its complementary sequences.

3. A plasmid, characterized for comprising a DNA fragment according to claim 1.

4. A host cell, characterized for being transformed with the plasmid according to claim 3, in order to allow the host cell to express the mentioned xylose/glucose active transporter.

5. The host cell according to claim 4, characterized for being a yeast.

6. The host cell according to claim 5, characterized for the yeast being Saccharonzyces cerevisiae.

7. Use of a host cell transformed according to claim 4, characterized for allowing the production of ethanol by means of xylose fermentation from a medium comprising a xylose source.


SEQUENCE LISTING (12 sequences)

SEQ ID NO: 1 - DNA, 1569 bp, C. intermedia
atgggtttggaggacaatagaatggttaagcgtttcgtcaacgttggcgagaagaaggctggctctactgccatggccatcatcgtcggtctttttgctgcttctggtggtgtccttttcggatacgatactggtactatttctggtgtgatgaccatggactacgttcttgctcgttacccttccaacaagcactcttttactgctgatgaatcttctttgattgtttctatcttgtctgttggtactttctttggtgcactttgtgctccattccttaacgacaccctcggtagacgttggtgtcttattctttctgctcttattgtcttcaacattggtgctatcttgcaggtcatctctactgccattccattgctttgtgctggtagagttattgcaggttttggtgtcggtttgatttctgctactattccattgtaccaatctgagactgctccaaagtggatcagaggtgccattgtctcttgttaccagtgggctattaccattggtcttttcttggcctcttgtgtcaacaagggtactgagcacatgactaactctggatcttacagaattccacttgctattcaatgtctttggggtcttatcttgggtatcggtatgatcttcttgccagagactccaagattctggatctccaagggtaaccaggagaaggctgctgagtctttggccagattgagaaagcttccaattgaccacccagactctctcgaggaattaagagacatcactgctgcttacgagttcgagactgtgtacggtaagtcctcttggagccaggtgttctctcacaagaaccaccagttgaagagattgttcactggtgtggctatccaggctttccagcaattgaccggtgttaacttcattttctactacggtactaccttcttcaagagagctggtgttaacggtttcactatctccttggccactaacattgtcaatgtcggttctactattccaggtattcttttgatggaagtcctcggtagaagaaacatgttgatgggtggtgctactggtatgtctctttctcaattgatcgttgccattgttggtgttgctacctcggaaaacaacaagtcttcccagtccgtccttgttgctttctcctgtattttcattgccttcttcgctgccacctggggtccatgtgcttgggttgttgttggtgagttgttcccattgagaaccagagctaagtctgtctccttgtgtactgcttccaactggttgtggaactggggtattgcttacgctactccatacatggtggatgaagacaagggtaacttgggttccaatgtgttcttcatctggggtggtttcaacttggcttgtgttttcttcgcttggtacttcatctacgagaccaagggtctttctttggagcaggtcgacgagttgtacgagcatgtcagcaaggcttggaagtctaagggcttcgttccatctaagcactctttcagagagcaggtggaccagcaaatggactccaaaactgaagctattatgtctgaagaagcttctgtttaa

SEQ ID NO: 2 - DNA, 22 bp, artificial
  primer ActCiL1
aacagagagaagatgacccaga

SEQ ID NO: 3 - DNA, 21 bp, artificial
  primer ActCiR1
gcaaagagaaaccagcgtaaa

SEQ ID NO: 4 - DNA, 23 bp, artificial
  primer CiGXSL1
ganganaanngnatggtnaanng

SEQ ID NO: 5 - DNA, 20 bp, artificial
  primer CiGXSL2
aanngnttngtnaangtngg

SEQ ID NO: 6 - DNA, 22 bp, artificial
  primer CiGXSR3
cgttaaggaatggagcacaaag

SEQ ID NO: 7 - DNA, 38 bp, artificial
  primer GXS1P1
atagcagatctcatatgggtttggaggacaatagaatg

SEQ ID NO: 8 - DNA, 39 bp, artificial
  primer GXS1P2
atagcagatcttctagattaaacagaagcrrcttcagac

SEQ ID NO: 9 - DNA, 28 bp, artificial
  primer HXT7prom1
aacctgcagctcgtaggaacaatttcgg

SEQ ID NO: 10 - DNA, 36 bp, artificial
  primer HXT7prom2
ggacgggacatatgctgattaaaattaaaaaaactt

SEQ ID NO: 11 - DNA, 38 bp, artificial
  primer PGK1term1
accgtgtctagataaattgaattgaattgaatcgatag

SEQ ID NO: 12 - DNA, 36 bp, artificial
  primer PGK1term2
taattagagctctcgaaagctttaacgaacgcagaa